(19) United States
(12) Patent Application Publication
(10) Pub. No.: US 2024/0108861 A1
(43) Pub. Date: Apr. 4, 2024

(54) VARIABLE DUROMETER DILATOR FOR INTRAVASCULAR ACCESS DEVICES

(71) Applicant: Cook Medical Technologies LLC, Bloomington, IN (US)

(72) Inventors: Georgios T. Hilas, Bloomington, IN (US); Quentin T. Hillen, Ellettsville, IN (US)

(21) Appl. No.: 18/480,258

(22) Filed: Oct. 3, 2023

Related U.S. Application Data
(60) Provisional application No. 63/412,946, filed on Oct. 4, 2022.

(51) Int. Cl. A61M 25/06; A61M 29/02

(52) U.S. Cl. CPC A61M 25/0662; A61M 29/02

(57) ABSTRACT

Disclosed herein is a medical device including an introducer sheath including a stiffness transition and a dilator including a stiffness transition. When the dilator occupies the lumen of the introducer sheath, the stiffness transition of the introducer sheath is advantageously offset longitudinally from the stiffness transition of the dilator. The medical device can navigate through a significantly higher number of turns in vasculature than medical devices without longitudinally offset stiffness transitions.

### RELATED APPLICATIONS

This application claims the benefit of U.S. Provisional Application No. 63/412,946, filed Oct. 4, 2022, which is hereby incorporated herein by reference in their entirety.

### FIELD

The present disclosure relates to medical devices. More particularly, the disclosure relates to introducer sheaths and dilators with offset transitions in stiffness.

### BACKGROUND

The statements in this section merely provide background information related to the present disclosure and may constitute prior art.

In today's medical field, many medical procedures require entry into a patent's blood vessel for purposes of accessing a desired site, for example, angioplasty and stenting. In order to gain access to the desired site, a sheath is usually advanced through the blood vessel. Once in place within the patient's vessel, various types of medical instrumentation can be fed through the sheath and positioned at the desired site so that the procedure may be performed.

To initially gain access to a particular site within a patient, a needle is used to puncture the patient's skin and gain entry to a desired blood vessel. A guide wire is then inserted into a lumen in the needle and is fed into the blood vessel. The needle is then removed, with the guide wire being left in place.

A dilator/sheath assembly is then placed over the guide wire and advanced to a position inside the blood vessel. The distal end of the dilator may be tapered to a relatively small diameter and extend beyond the distal end of the sheath. The tapered distal end of the dilator allows the dilator/sheath assembly to be introduced into a patient's vessel in a manner that gradually increases the size of the opening in the vessel so that the vessel can ultimately accommodate the larger sized sheath without causing trauma, injury, or other difficulties to the patient. Once the guide wire and the dilator/sheath assembly are advanced within the blood vessel to the desired site, the dilator is removed.

### SUMMARY

In an example, the present disclosure provides a medical device. The medical device includes an introducer sheath. The introducer sheath includes a tubular structure. The tubular structure includes a wall defining a sheath lumen extending longitudinally throughout the tubular structure. The tubular structure defines a longitudinal axis therethrough. The tubular structure further includes a first longitudinal sheath section having a first sheath stiffness; and a second longitudinal sheath section distal to the first longitudinal sheath section, the second longitudinal sheath section having a second sheath stiffness that is less than the first sheath stiffness; and a sheath transition from the first longitudinal sheath section to the second longitudinal sheath section. The medical device further includes a dilator configured to occupy the sheath lumen. The dilator includes a shaft including a wall defining a dilator lumen extending longitudinally throughout the shaft, the shaft defining a second longitudinal axis that is coaxial with the longitudinal axis when the dilator occupies the sheath lumen. The shaft further includes a first longitudinal dilator section having a first dilator stiffness; and a second longitudinal dilator section distal to the first longitudinal dilator section, the second longitudinal dilator section having a second dilator stiffness that is less than the first dilator stiffness; and a dilator transition from the first longitudinal dilator section to the second longitudinal dilator section. The sheath transition is offset longitudinally from the dilator transition when the dilator occupies the sheath lumen. In certain examples, the first sheath stiffness may be less than the first dilator stiffness. In other examples, the sheath transition may be distal to the dilator transition. In still other examples, the second dilator stiffness may be less than the first sheath stiffness and greater than the second sheath stiffness. In still other examples, the dilator transition may be distal to the sheath transition. In still other examples, the second dilator stiffness may be less than the second sheath stiffness. In still other examples, the second longitudinal sheath section may include a distal sheath tip at a distal end of the second longitudinal sheath section; and when the dilator occupies the sheath lumen, the second longitudinal dilator section may be configured to extend distally at least one centimeter through the distal sheath tip. In still other examples, the shaft may further include a third longitudinal dilator section distal to the second longitudinal dilator section, the third longitudinal dilator section having a third dilator stiffness different from the second dilator stiffness. In still other examples, the third dilator stiffness may be less than the second dilator stiffness. In still other examples, the third dilator stiffness may be greater than the second dilator stiffness and/or equal to the first dilator stiffness. In still other examples, a longitudinal dilator length may be greater than a longitudinal sheath length.

In another example, the present disclosure provides an introducer sheath. The introducer sheath includes a wall defining a sheath lumen extending longitudinally throughout the tubular structure, the tubular structure defining a longitudinal axis therethrough. The tubular structure includes a first longitudinal sheath section having a first sheath stiffness; a second longitudinal sheath section distal to the first longitudinal sheath section, the second longitudinal sheath section having a second sheath stiffness that is less than the first sheath stiffness; and a sheath transition from the first longitudinal sheath section to the second longitudinal sheath section. A dilator is configured to occupy the sheath lumen such that the sheath transition is offset longitudinally from a dilator transition from a first longitudinal dilator section to a second longitudinal dilator section. The dilator includes a shaft, the shaft including a wall defining a dilator lumen extending longitudinally throughout the shaft; the first longitudinal dilator section having a first dilator stiffness; and the second longitudinal dilator section distal to the first longitudinal dilator section and having a second dilator stiffness that is less than the first dilator stiffness. In certain examples, the first sheath stiffness may be less than the first dilator stiffness. In other examples, the sheath transition may be configured to be distal to the dilator transition. In still other examples, the second dilator stiffness may be less than the first sheath stiffness and greater than the second sheath stiffness. In still other examples, the dilator transition may be configured to be distal to the sheath transition.

In yet another example, the present disclosure provides a dilator. The dilator includes a shaft. The shaft includes a wall defining a dilator lumen extending longitudinally throughout the shaft; a first longitudinal dilator section having a first dilator stiffness; a second longitudinal dilator section distal to the first longitudinal dilator section, the second longitudinal dilator section having a second dilator stiffness that is less than the first dilator stiffness; and a dilator transition from the first longitudinal dilator section to the second longitudinal dilator section. The dilator is configured to occupy a sheath lumen of an introducer sheath such that a sheath transition from a first longitudinal sheath section to a second longitudinal sheath section is offset longitudinally from the dilator transition. The introducer sheath includes a tubular structure including a wall defining the sheath lumen extending longitudinally throughout the tubular structure, the tubular structure defining a longitudinal axis therethrough, the tubular structure including the first longitudinal sheath section having a first sheath stiffness, and the second longitudinal sheath section distal to the first longitudinal sheath section and having a second sheath stiffness that is less than the first sheath stiffness. In certain examples, the first sheath stiffness may be less than first dilator stiffness. In other examples, the sheath transition may be configured to be distal to the dilator transition. In still other examples, the second dilator stiffness may be less than the first sheath stiffness and greater than the second sheath stiffness.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

In order that the present disclosure may be well understood, there will now be described various forms thereof, given by way of example, reference being made to the accompanying drawings. The components in the figures are not necessarily to scale. Moreover, in the figures, like-referenced numerals designate corresponding parts through the different views.

FIG. 1 illustrates a side view of an example of an introducer sheath, according to the principles of the present disclosure;

FIG. 2 illustrates a sectional view of a distal tip of the example of an introducer sheath of FIG. 1;

FIG. 3 illustrates a side view of an example of a dilator, according to the principles of the present disclosure;

FIG. 4 illustrates a side view of another example of a dilator, according to the principles of the present disclosure;

FIG. 5 illustrates a distal end view of the example of a dilator of FIG. 4;

FIG. 6 illustrates a model tracking path used to analyze various medical devices for their abilities to complete turns in vasculature without device failure;

FIG. 7 illustrates an exploded view of the model tracking path in FIG. 6 indicating the number of turns in vasculature at each point along the model tracking path;

FIG. 8 illustrates a side view of an example of an introducer sheath and an example of a dilator, shown separately, according to the principles of the present disclosure; and

FIG. 9 illustrates a side view of an example of an introducer sheath and an example of a dilator, the dilator occupying the sheath lumen, according to the principles of the present disclosure.

The drawings described herein are for illustration purposes only and are not intended to limit the scope of the present disclosure in any way.

### DETAILED DESCRIPTION

The following description presents examples and is not intended to limit the present disclosure, application, or uses. It should be understood that throughout the drawings, corresponding reference numerals indicate like or corresponding parts and features.

In adding reference denotations to elements of each drawing, although the same elements are displayed on a different drawing, it should be noted that the same elements have the same denotations. In addition, in describing one aspect of the present disclosure, if it is determined that a detailed description of related well-known configurations or functions blurs the gist of one aspect of the present disclosure, it will be omitted.

In the following discussion, the terms “proximal” and “distal” will be used to describe the opposing axial ends of the device, as well as the axial ends of various component features. The term “proximal” is used in its conventional sense to refer to the end of the device (or component) that is closest to the medical professional during use of the assembly. The term “distal” is used in its conventional sense to refer to the end of the device (or component) that is initially inserted into the patient, or that is closest to the patient during use. The term “longitudinal” will be used to refer to an axis that aligns with the proximal-distal axis of the device (or component). The terms “radially” and “radial” will be used to refer to elements, surfaces, or assemblies relative to one another that may extend perpendicularly from a longitudinal axis. The term “circumference,” “circumferentially,” and “circumferential” will be used to refer to elements, surfaces, or assemblies relative to one another encircling a longitudinal axis at a radius.

The uses of the terms “a” and “an” and “the” and similar referents in the context of describing the present disclosure (especially in the context of the following claims) are to be construed to cover both the singular and the plural, unless otherwise indicated herein or clearly contradicted by context. The use of the term “plurality of” is defined by the Applicant in the broadest sense, superseding any other implied definitions or limitations hereinbefore or hereinafter unless expressly asserted by Applicant to the contrary, to mean a quantity of more than one. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, the terms “comprise(s),” “include(s),” “having,” “has,” “can,” “contain(s),” and variants thereof, are intended to be open-ended transitional phrases, terms, or words that do not preclude the possibility of additional acts or structures. The present description also contemplates other examples “comprising,” “consisting of,” and “consisting essentially of,” the examples or elements presented herein, whether explicitly set forth or not.

In describing elements of the present disclosure, the terms 1^{st}, 2^{nd}, first, second, A, B, (a), (b), and the like may be used herein. These terms are only used to distinguish one element from another element, but do not limit the corresponding elements irrespective of the nature or order of the corresponding elements.

Unless otherwise stated, all terms used herein, including technical or scientific terms, have the same meanings as those generally understood by those skilled in the art to which the present disclosure pertains. Such terms as those defined in a generally used dictionary are to be interpreted as having meanings equal to the contextual meanings in the relevant field of art.

As used herein, the term “about,” when used in the context of a numerical value or range set forth means a variation of ±15%, or less, of the numerical value. For example, a value differing by ±15%, ±14%, ±10%, or ±5%, among others, would satisfy the definition of “about,” unless more narrowly defined in particular instances.

As used herein, the term “stiffness” refers to a measure of a hardness of a material, such as a polymer, elastomer, or rubber. The term is relative and unitless, but higher stiffness indicates a greater resistance to indentation and thus a harder material that is less flexible and bendable, while a lower relative stiffness indicates a lower resistance to indentation and thus a softer material that is more flexible and bendable. Methods for measuring or otherwise determining relative stiffness of materials are generally known to those of ordinary skill in the art. In a particular example, stiffness may be measured by a Shore durometer, which measures the depth of an indentation in a material created by a given force on a standardized presser foot. The depth may be dependent on the hardness of the material, the viscoelastic properties of the material, the shape of the presser foot, and the duration of the test There are several scales of durometer, which may be used for materials with different properties, but the two most common scales are the ASTM D2240 type A scale, in which a presser foot indents the material for 15 seconds at a force of about 10 N, and the type D scale, in which the presser foot penetrates the surface of the material for 15 seconds at a force of about 50 N. Each durometer scale results in a value between 0 and 100, with increasing values indicating a harder material.

Referring to FIG. 1, a side view of an example of an introducer sheath 100 is illustrated. Introducer sheath 100 includes tubular structure 104 including a wall 118 that defines a sheath lumen 120 extending longitudinally throughout tubular structure 104 from proximal end 114 to distal tip 112. Tubular structure 104 defines a first longitudinal axis 122 extending therethrough. Proximal end 114 of tubular structure 104 is secured to valve body 102. Tubular structure 104 includes first longitudinal sheath section 106 and second longitudinal sheath section 108. Second longitudinal sheath section 108 is distal to first longitudinal sheath section 106. First longitudinal sheath section 106 has a first sheath stiffness. Second longitudinal sheath section 106 has a second sheath stiffness that is less than the first sheath stiffness. First longitudinal sheath section 106 and second longitudinal sheath section 108 are connected at sheath transition 110, and tubular structure 104 may be made by butt-bonding first longitudinal sheath section 106 to second longitudinal sheath section 108. Alternatively, second longitudinal sheath section 108 may be integral to first longitudinal sheath section 106, and tubular structure 104 may be formed by a process such as total intermittent extrusion in which tubular structure 104 is prepared such that first longitudinal sheath section 106 has first sheath stiffness and second longitudinal sheath section 108 has second sheath stiffness. Tubular structure 104 has a length L_{S} from proximal end 114 to distal tip 112. Second longitudinal sheath section 108 has a length that is the sum of L_{A}, the length of intermediate longitudinal segment 116, and L_{B}, the longitudinal length of distal tip 112. First longitudinal sheath section 106 has a length that is the length L_{S} of tubular structure 104 minus the sum of L_{A}+L_{B}, or L_{S}−(L_{A}+L_{B}).

In certain examples, the first sheath stiffness and the second sheath stiffness may differ on the ASTM D2240 type D scale for measurement by a Shore durometer by a value of at least 5D, or at least 10D, or at least 15D, or at least 20D, or at least 25D, or at least 30D, or at least 35D, or at least 40D, or at least 45D, or at least 50D, or at least 55D, or at least 60D, or at least 65D, or at least 70D, or at least 75D, or at least 80D, or at least 85D, or at least 90D, or at least 95D. In particular examples, the first sheath stiffness may be greater than 50D, or greater than 55D, or greater than 60D, or greater than 65D, or greater than 70D, or greater than 75D, and the second sheath stiffness may be less than 50D, or less than 45D, or less than 40D, or less than 35D, or less than 30D, or less than 25D. In other particular examples, the first sheath stiffness may be about 52D, or about 54D, or about 56D, or about 58D, or about 60D, or about 62D, or about 64D, or about 66D, or about 68D, or about 70D, or about 72D, or about 74D, or about 76D, or about 78D, or about 80D, and the second sheath stiffness may be about 48D, or about 46D, or about 44D, or about 42D, or about 40D, or about 38D, or about 36D, or about 34D, or about 32D, or about 30D, or about 28D, or about 26D, or about 24D, or about 22D, or about 20D.

In certain examples, length L_{S} of tubular structure 104 may be at least about 20 cm, or at least about 22 cm, or at least about 24 cm, or at least about 26 cm, or at least about 28 cm, or at least about 30 cm, or at least about 32 cm, or at least about 34 cm, or at least about 36 cm, or at least about 38 cm, or at least about 40 cm, or at least about 42 cm, or at least about 44 cm, or at least about 46 cm, or at least about 48 cm, or at least about 50 cm, or at least about 52 cm, or at least about 54 cm, or at least about 56 cm, or at least about 58 cm, or at least about 60 cm. In other particular examples, the longitudinal length L_{B} of distal tip 112 may be at least about 0.2 cm, or at least about 0.3 cm, or at least about 0.4 cm, or at least about 0.5 cm, or at least about 0.6 cm, or at least about 0.7 cm, or at least about 0.8 cm, or at least about 0.9 cm, or at least about 1.0 cm, or at least about 1.1 cm, or at least about 1.2 cm, or at least about 1.3 cm, or at least about 1.4 cm, or at least about 1.5 cm.

In an example, tubular structure 104 may be of a longitudinal cross-sectional size within a range of sizes of from 4 French (“Fr.”) to 24 Fr. As will be understood to those of skill in the art, the French scale or French gauge system commonly used to measure the size of a medical device such as a catheter refers to three times the length, measured in millimeters (“mm”), of the outer diameter of the medical device. In the case of tubular structure 104, the French gauge system would refer to three times the length, measured in millimeters, from wall 118 to wall 118 of tubular structure 104. For example, a round tubular structure 104 of 1 French will have an outer diameter of ⅓ millimeters; a round catheter of 3 French will have an outer diameter of 1 millimeter. In certain examples, tubular structure 104 may have a size of from 4 French to 24 French, including from 5 French, or from 6 French, or from 7 French, or from 8 French, or from 9 French, or from 10 French, or from 11 French, or from 12 French, or from 13 French, or from 14 French, or from 15 French, or from 16 French, or from 17 French, or from 18 French, or from 19 French, or from 20 French, or from 21 French, or from 22 French, or from 23 French to 24 French; or from 4 French to 5 French, or to 6 French, or to 7 French, or to 8 French, or to 9 French, or to 10 French, or to 11 French, or to 12 French, or to 13 French, or to 14 French, or to 15 French, or to 16 French, or to 17 French, or to 18 French, or to 19 French, or to 20 French, or to 21 French, or to 22 French, or to 23 French, or to 24 French; or any other range of from one of the above minima to one of the above maxima.

In an example, first longitudinal sheath section 106 and second longitudinal sheath section 108 may each include a blend of 0-95% polypropylene (“PP”) impact copolymer and 5-100% high performance elastomer. Examples of high performance elastomers may include thermoplastic elastomers such as thermoplastic olefin (“TPO”), thermoplastic polyurethane (“TPU”), thermoplastic vulcanizate (“TPV”), polyester elastomer, polyamide elastomers such as polyester block amide (“PEBA”), nylon, polyethylene, and fluoropolymers such as fluorinated ethylene propylene (“FEP”), perfluoroalkoxy alkane (“PFA”), and ethylene tetrafluoroethylene (“ETFE”).

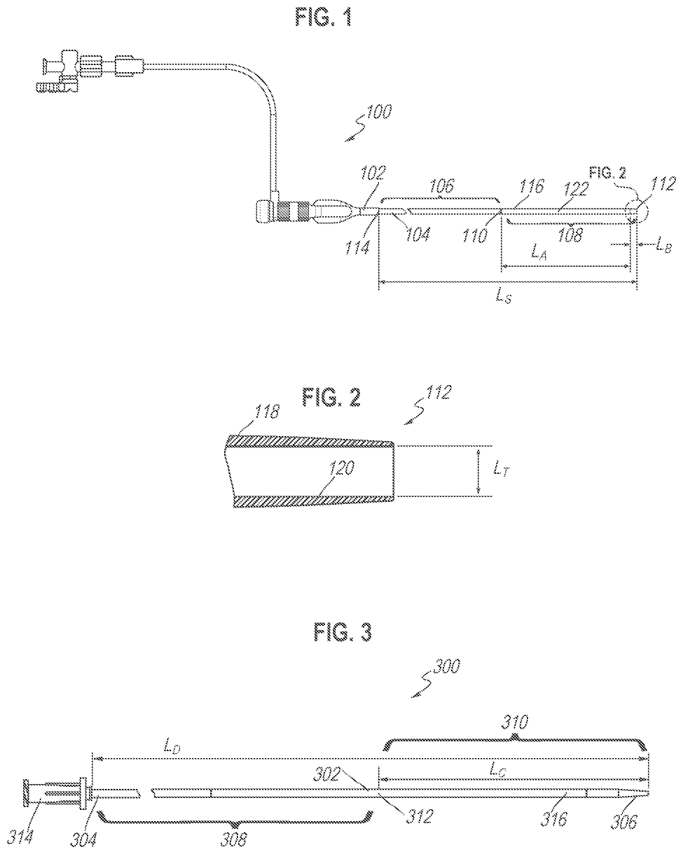

Referring to FIG. 2, a sectional view of distal tip 112 of the example of introducer sheath 100 is illustrated. As shown in FIG. 2, wall 118 may slightly taper towards the distal end of tubular structure 104. Sheath lumen 120 of tubular structure 104 may have a diameter of L_{T}. Diameter L_{T} may be of a size such that a dilator may reversibly occupy sheath lumen 120.

Referring to FIG. 3, a side view of an example of a dilator 300 is illustrated. Dilator 300 includes shaft 302 that includes a wall defining a dilator lumen that extends longitudinally throughout shaft 302. Shaft 302 defines a second longitudinal axis 316. Second longitudinal axis 316 is coaxial with first longitudinal axis 122 of introducer sheath 100 when dilator 300 occupies sheath lumen 120 of introducer sheath 100. Shaft 302 extends from proximal end 304 to distal tip 306. Shaft 302 includes first longitudinal dilator section 308 and second longitudinal dilator section 310. Second longitudinal dilator section 310 is distal to first longitudinal dilator section 308. First longitudinal dilator section 308 has a first dilator stiffness. Second longitudinal dilator section 310 has a second dilator stiffness that is less than the first dilator stiffness. First longitudinal dilator section 308 and second longitudinal dilator section 310 are connected at dilator transition 312, and shaft 302 may be made by butt-bonding first longitudinal dilator section 308 to second longitudinal dilator section 310. Alternatively, second longitudinal dilator section 310 may be integral to first longitudinal dilator section 308, and shaft 302 may be formed by a process such as total intermittent extrusion in which shaft 302 is prepared such that first longitudinal dilator section 308 has first dilator stiffness and second longitudinal dilator section 310 has second dilator stiffness. Proximal end 304 is attached to dilator hub 314. Shaft 302 has a length L_{D} from proximal end 304 to distal tip 306. Second longitudinal dilator section 310 has a length L_{C}. Length L_{C} may be greater than the sum of L_{A}+L_{B}. Alternatively, length L_{C} may be less than the sum of L_{A}+L_{B}. First longitudinal dilator section 310 has a length of L_{D} of shaft 302 minus the length L_{C} of second longitudinal section 310. When dilator 300 occupies sheath lumen 120 of introducer sheath 100, sheath transition 110 is offset longitudinally from dilator transition 312, and sheath transition 110 is proximal or distal to dilator transition 312.

In an example, when dilator 300 occupies sheath lumen 120 of introducer sheath 100, sheath transition 110 is offset longitudinally from dilator transition 312 by a distance of at least about 0.1 cm, or at least about 0.2 cm, or at least about 0.3 cm, or at least about 0.4 cm, or at least about 0.5 cm, or at least about 0.6 cm, or at least about 0.7 cm, or at least about 0.8 cm, or at least about 0.9 cm, or at least about 1.0 cm, or at least about 1.1 cm, or at least about 1.2 cm, or at least about 1.3 cm, or at least about 1.4 cm, or at least about 1.5 cm, or at least about 1.6 cm, or at least about 1.7 cm, or at least about 1.8 cm, or at least about 1.9 cm, or at least about 2.0 cm, or at least about 2.1 cm, or at least about 2.2 cm, or at least about 2.3 cm, or at least about 2.4 cm, or at least about 2.5 cm, or at least about 2.6 cm, or at least about 2.7 cm, or at least about 2.8 cm, or at least about 2.9 cm, or at least about 3.0 cm, or at least about 3.1 cm, or at least about 3.2 cm, or at least about 3.3 cm, or at least about 3.4 cm, or at least about 3.5 cm, or at least about 3.6 cm, or at least about 3.7 cm, or at least about 3.8 cm, or at least about 3.9 cm, or at least about 4.0 cm, or at least about 4.1 cm, or at least about 4.2 cm, or at least about 4.3 cm, or at least about 4.4 cm, or at least about 4.5 cm, or at least about 4.6 cm, or at least about 4.7 cm, or at least about 4.8 cm, or at least about 4.9 cm, or at least about 5.0 cm, or at least about 5.1 cm, or at least about 5.2 cm, or at least about 5.3 cm, or at least about 5.4 cm, or at least about 5.5 cm, or at least about 5.6 cm, or at least about 5.7 cm, or at least about 5.8 cm, or at least about 5.9 cm, or at least about 6.0 cm, or at least about 6.1 cm, or at least about 6.2 cm, or at least about 6.3 cm, or at least about 6.4 cm, or at least about 6.5 cm, or at least about 6.6 cm, or at least about 6.7 cm, or at least about 6.8 cm, or at least about 6.9 cm, or at least about 7.0 cm, or at least about 7.1 cm, or at least about 7.2 cm, or at least about 7.3 cm, or at least about 7.4 cm, or at least about 7.5 cm, or at least about 7.6 cm, or at least about 7.7 cm, or at least about 7.8 cm, or at least about 7.9 cm, or at least about 8.0 cm, or at least about 8.1 cm, or at least about 8.2 cm, or at least about 8.3 cm, or at least about 8.4 cm, or at least about 8.5 cm, or at least about 8.6 cm, or at least about 8.7 cm, or at least about 8.8 cm, or at least about 8.9 cm, or at least about 9.0 cm, or at least about 9.1 cm, or at least about 9.2 cm, or at least about 9.3 cm, or at least about 9.4 cm, or at least about 9.5 cm, or at least about 9.6 cm, or at least about 9.7 cm, or at least about 9.8 cm, or at least about 9.9 cm, or at least about 10.0 cm.

In certain examples, the first dilator stiffness and the second dilator stiffness may differ on the ASTM D2240 type D scale for measurement by a Shore durometer by a value of at least 5D, or at least 10D, or at least 15D, or at least 20D, or at least 25D, or at least 30D, or at least 35D, or at least 40D, or at least 45D, or at least 50D, or at least 55D, or at least 60D, or at least 65D, or at least 70D, or at least 75D, or at least 80D, or at least 85D, or at least 90D, or at least 95D. In particular examples, the first dilator stiffness may be greater than the first sheath stiffness and greater than 55D, or greater than 60D, or greater than 65D, or greater than 70D, or greater than 75D. In other particular examples, when shaft 302 of dilator 300 occupies sheath lumen 120 of introducer sheath 100 and dilator transition 312 is proximal to sheath transition 110, the second dilator stiffness may be greater than the second sheath stiffness but less than the first sheath stiffness. In still other particular examples, when shaft 302 of dilator 300 occupies sheath lumen 120 of introducer sheath 100 and dilator transition 312 is distal to sheath transition 110, the second dilator stiffness may be less than the second sheath stiffness, and may be less than 45D, or less than 40D, or less than 35D, or less than 30D, or less than 25D. In still other particular examples, the first dilator stiffness may be about 56D, or about 58D, or about 60D, or about 62D, or about 64D, or about 66D, or about 68D, or about 70D, or about 72D, or about 74D, or about 76D, or about 78D, or about 80D, and the second dilator stiffness may be about 55D, or about 54D, or about 52D, or about 50D, or about 48D, or about 46D, or about 44D, or about 42D, or about 40D, or about 38D, or about 36D, or about 34D, or about 32D, or about 30D, or about 28D, or about 26D, or about 24D, or about 22D, or about 20D.

By including a single stiffness transition in dilator 300 and a single stiffness transition in introducer sheath 100, and offsetting longitudinally dilator transition 312 from sheath transition 110, the offset stiffness transitions advantageously result in the combined effect of a series of stiffness transitions. The combined series of stiffness transitions range from highest stiffness in first longitudinal dilator section 308 to a lower stiffness in first longitudinal sheath section 106 to a lower stiffness in second longitudinal dilator section 310 (or second longitudinal sheath section 108), and finally to lowest stiffness in second longitudinal sheath section 108 (or second longitudinal dilator section 310). The offset stiffness transitions result in an advantageously unique transition in feel for an operator of an introducer sheath 100 and dilator 300 while minimizing stiffness transitions in each of introducer sheath 100 and dilator 300.

In certain examples, length L_{D} of shaft 302 may be at least about 0.5 cm longer than length L_{S} of tubular structure 104, or at least about 1.0 cm longer, or at least about 1.5 cm longer, or at least about 2.0 cm longer, or at least about 2.5 cm longer, or at least about 3.0 cm longer, or at least about 3.5 cm longer, or at least about 4.0 cm longer, or at least about 4.5 cm longer, or at least about 5.0 cm longer, or at least about 5.5 cm longer, or at least about 6.0 cm longer, or at least about 6.5 cm longer, or at least about 7.0 cm longer, or at least about 7.5 cm longer, or at least about 8.0 cm longer, or at least about 8.5 cm longer, or at least about 9.0 cm longer, or at least about 9.5 cm longer, or at least about 10.0 cm longer than length L_{S}. In other examples, when shaft 302 of dilator 300 occupies sheath lumen 120 of introducer sheath 100, the distal end of distal tip 306 of dilator 300 may extend at least about 0.5 cm beyond the distal end of distal tip 112 of introducer sheath 100, or may extend at least about 1.0 cm, or at least about 1.5 cm, or at least about 2.0 cm, or at least about 2.5 cm, or at least about 3.0 cm, or at least about 3.5 cm, or at least about 4.0 cm, or at least about 4.5 cm, or at least about 5.0 cm.

In certain examples, shaft 302 of dilator 300 may be of a longitudinal cross-sectional size such that shaft 302 may reversibly occupy sheath lumen 120 of introducer sheath 100.

In an example, first longitudinal dilator section 308 and second longitudinal dilator section 310 may each include a blend of 0-95% polypropylene (PP) impact copolymer and 5-100% high performance elastomer. Examples of high performance elastomers may include thermoplastic elastomers such as thermoplastic olefin (TPO), thermoplastic polyurethane (TPU), thermoplastic vulcanizate (TPV), polyester elastomer, polyamide elastomers such as polyester block amide (PEBA), nylon, polyethylene, and fluoropolymers such as fluorinated ethylene propylene (FEP), perfluoroalkoxy alkane (PFA), and ethylene tetrafluoroethylene (ETFE).

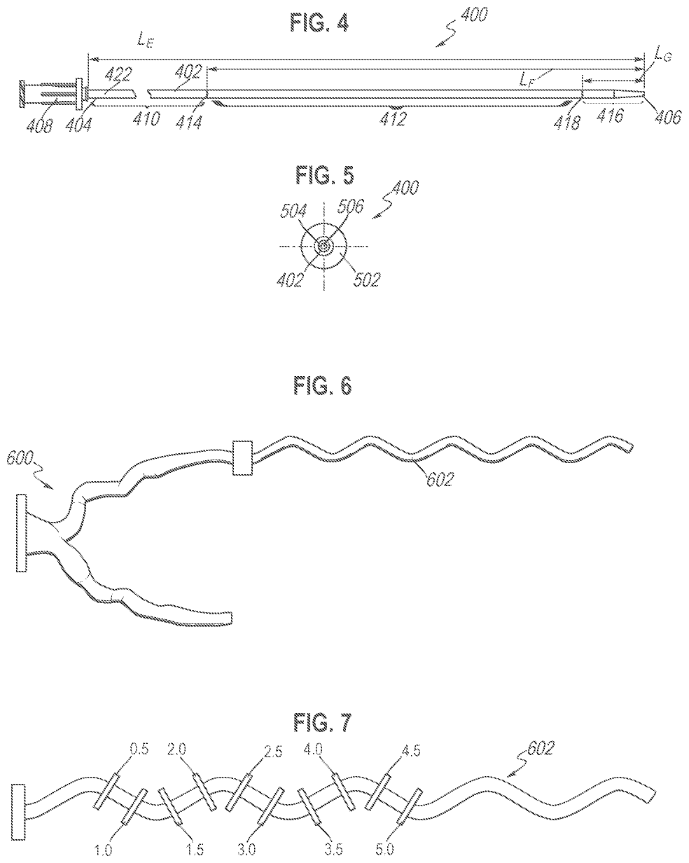

Referring to FIG. 4, a side view of another example of a dilator 400 is illustrated. Dilator 400 includes shaft 402 that includes a wall defining a dilator lumen that extends longitudinally throughout shaft 402. Shaft 402 defines a second longitudinal axis 422. Second longitudinal axis 422 is coaxial with first longitudinal axis 122 of introducer sheath 100 when dilator 400 occupies sheath lumen 120 of introducer sheath 100. Shaft 402 extends from proximal end 404 to distal tip 406. Shaft 402 includes first longitudinal dilator section 410, second longitudinal dilator section 412, and third longitudinal dilator section 416. Second longitudinal dilator section 412 is distal to first longitudinal dilator section 410. Third longitudinal dilator section 416 is distal to second longitudinal dilator section 412. First longitudinal dilator section 410 has a first dilator stiffness. Second longitudinal dilator section 412 has a second dilator stiffness that is less than the first dilator stiffness. Third longitudinal dilator section 416 has a third dilator stiffness that is different than the second dilator stiffness. First longitudinal dilator section 410 and second longitudinal dilator section 412 are connected at first dilator transition 414. Second longitudinal dilator section 412 and third longitudinal dilator section 416 are connected at second dilator transition 418. Shaft 402 may be made by butt-bonding first longitudinal dilator section 410 to second longitudinal dilator section 412 and second longitudinal dilator section 412 to third longitudinal dilator section 416. Alternatively, second longitudinal dilator section 412 may be integral to first longitudinal dilator section 410, and third longitudinal dilator section 416 may be integral to second longitudinal dilator section 412, and shaft 402 may be formed by a process such as total intermittent extrusion in which shaft 402 is prepared such that first longitudinal dilator section 410 has first dilator stiffness, second longitudinal dilator section 412 has second dilator stiffness, and third longitudinal dilator section 416 has third dilator stiffness. Proximal end 404 is attached to dilator hub 408. Shaft 402 has a length L_{E} from proximal end 404 to distal tip 406. Length L_{E} may be longer than a length of tubular structure 104 of introducer sheath 100 by at least about 0.5 cm, or at least about 1.0 cm, or at least about 1.5 cm, or at least about 2.0 cm, or at least about 2.5 cm, or at least about 3.0 cm, or at least about 3.5 cm, or at least about 4.0 cm, or at least about 4.5 cm, or at least about 5.0 cm, or at least about 5.5 cm, or at least about 6.0 cm, or at least about 6.5 cm, or at least about 7.0 cm, or at least about 7.5 cm, or at least about 8.0 cm, or at least about 8.5 cm, or at least about 9.0 cm, or at least about 9.5 cm, or at least about 10.0 cm.

Third longitudinal dilator section 416 has a longitudinal length L_{G}. When dilator 400 occupies sheath lumen 120 of introducer sheath 100, distal tip 406 of dilator 400 may extend beyond the distal tip of the introducer sheath by greater than about length L_{G}, less than about length L_{G}, or equal to about length L_{G}, and the longitudinal distance by which distal tip 406 of dilator 400 extends beyond the distal tip of the introducer sheath may correspond to at least about 0.5 cm, or at least about 1.0 cm, or at least about 1.5 cm, or at least about 2.0 cm, or at least about 2.5 cm, or at least about 3.0 cm, or at least about 3.5 cm, or at least about 4.0 cm, or at least about 4.5 cm, or at least about 5.0 cm.

Second longitudinal dilator section 412 and third longitudinal dilator section 416 have a combined longitudinal length L_{F} Second longitudinal dilator section 412 has a longitudinal length corresponding to L_{F}−L_{G}. Longitudinal length L_{F}−L_{G} of second longitudinal dilator section 412 may be such that when dilator 400 occupies sheath lumen 120 of introducer sheath 100, sheath transition 110 is offset longitudinally from each of first dilator transition 414 and second dilator transition 418. Sheath transition 110 may be offset longitudinally from first dilator transition 414, and additionally, or alternatively, sheath transition 110 may be offset longitudinally from second dilator transition 418, by a distance of at least about 0.1 cm, or at least about 0.2 cm, or at least about 0.3 cm, or at least about 0.4 cm, or at least about 0.5 cm, or at least about 0.6 cm, or at least about 0.7 cm, or at least about 0.8 cm, or at least about 0.9 cm, or at least about 1.0 cm, or at least about 1.1 cm, or at least about 1.2 cm, or at least about 1.3 cm, or at least about 1.4 cm, or at least about 1.5 cm, or at least about 1.6 cm, or at least about 1.7 cm, or at least about 1.8 cm, or at least about 1.9 cm, or at least about 2.0 cm, or at least about 2.1 cm, or at least about 2.2 cm, or at least about 2.3 cm, or at least about 2.4 cm, or at least about 2.5 cm, or at least about 2.6 cm, or at least about 2.7 cm, or at least about 2.8 cm, or at least about 2.9 cm, or at least about 3.0 cm, or at least about 3.1 cm, or at least about 3.2 cm, or at least about 3.3 cm, or at least about 3.4 cm, or at least about 3.5 cm, or at least about 3.6 cm, or at least about 3.7 cm, or at least about 3.8 cm, or at least about 3.9 cm, or at least about 4.0 cm, or at least about 4.1 cm, or at least about 4.2 cm, or at least about 4.3 cm, or at least about 4.4 cm, or at least about 4.5 cm, or at least about 4.6 cm, or at least about 4.7 cm, or at least about 4.8 cm, or at least about 4.9 cm, or at least about 5.0 cm, or at least about 5.1 cm, or at least about 5.2 cm, or at least about 5.3 cm, or at least about 5.4 cm, or at least about 5.5 cm, or at least about 5.6 cm, or at least about 5.7 cm, or at least about 5.8 cm, or at least about 5.9 cm, or at least about 6.0 cm, or at least about 6.1 cm, or at least about 6.2 cm, or at least about 6.3 cm, or at least about 6.4 cm, or at least about 6.5 cm, or at least about 6.6 cm, or at least about 6.7 cm, or at least about 6.8 cm, or at least about 6.9 cm, or at least about 7.0 cm, or at least about 7.1 cm, or at least about 7.2 cm, or at least about 7.3 cm, or at least about 7.4 cm, or at least about 7.5 cm, or at least about 7.6 cm, or at least about 7.7 cm, or at least about 7.8 cm, or at least about 7.9 cm, or at least about 8.0 cm, or at least about 8.1 cm, or at least about 8.2 cm, or at least about 8.3 cm, or at least about 8.4 cm, or at least about 8.5 cm, or at least about 8.6 cm, or at least about 8.7 cm, or at least about 8.8 cm, or at least about 8.9 cm, or at least about 9.0 cm, or at least about 9.1 cm, or at least about 9.2 cm, or at least about 9.3 cm, or at least about 9.4 cm, or at least about 9.5 cm, or at least about 9.6 cm, or at least about 9.7 cm, or at least about 9.8 cm, or at least about 9.9 cm, or at least about 1.0 cm.

First longitudinal dilator section 410 has a longitudinal length corresponding to L_{E}-L_{F} and may be of such a length such that when dilator 400 occupies sheath lumen 120 of introducer sheath 100, distal surface 502 of dilator hub 408 may confront or approximate proximal end 122 of valve body 102 of introducer sheath 100.

In certain examples, the first dilator stiffness may differ from the second dilator stiffness on the ASTM D2240 type D scale for measurement by a Shore durometer by a value of at least 5D, or at least 10D, or at least 15D, or at least 20D, or at least 25D, or at least 30D, or at least 35D, or at least 40D, or at least 45D, or at least 50D, or at least 55D, or at least 60D, or at least 65D, or at least 70D, or at least 75D, or at least 80D, or at least 85D, or at least 90D, or at least 95D. In particular examples, the first dilator stiffness may be greater than the first sheath stiffness and greater than 55D, or greater than 60D, or greater than 65D, or greater than 70D, or greater than 75D. In other particular examples, when shaft 402 of dilator 400 occupies sheath lumen 120 of introducer sheath 100 and first dilator transition 414 is proximal to sheath transition 110, the second dilator stiffness may be greater than the second sheath stiffness but less than the first sheath stiffness. In still other particular examples, when shaft 402 of dilator 400 occupies sheath lumen 120 of introducer sheath 100 and first dilator transition 414 is distal to sheath transition 110, the second dilator stiffness may be less than the second sheath stiffness, and may be less than 45D, or less than 40D, or less than 35D, or less than 30D, or less than 25D. In still other particular examples, the first dilator stiffness may be about 56D, or about 58D, or about 60D, or about 62D, or about 64D, or about 66D, or about 68D, or about 70D, or about 72D, or about 74D, or about 76D, or about 78D, or about 80D, and the second dilator stiffness may be about 55D, or about 54D, or about 52D, or about 50D, or about 48D, or about 46D, or about 44D, or about 42D, or about 40D, or about 38D, or about 36D, or about 34D, or about 32D, or about 30D, or about 28D, or about 26D, or about 24D, or about 22D, or about 20D. In still other particular examples, the third dilator stiffness may be less than the second dilator stiffness. In still other particular examples, the third dilator stiffness is greater than the second dilator stiffness and/or equal to the first dilator stiffness.

Whether first dilator transition 414 is distal or proximal to sheath transition 110 when dilator 400 occupies sheath lumen 120 of introducer sheath 100, by limiting the stiffness transitions in dilator 400 and including only a single stiffness transition in introducer sheath 100 and offsetting longitudinally from each other first dilator transition 414, sheath transition 110, and second dilator transition 418, advantageously the offset stiffness transitions result in the combined effect of a series of stiffness transitions from highest stiffness in first longitudinal dilator section 410 to a lower stiffness in first longitudinal sheath section 106, to a lower stiffness in second longitudinal dilator section 412 (or second longitudinal sheath section 108), and finally to lowest stiffness in second longitudinal sheath section 108 (or second longitudinal dilator section 412, or third longitudinal dilator section 416). The offset stiffness transitions result in an advantageously unique transition in feel for an operator of an introducer sheath 100 and dilator 400 while minimizing stiffness transitions in each of introducer sheath 100 and dilator 400.

In certain examples, shaft 402 of dilator 400 may be of a longitudinal cross-sectional size such that shaft 402 may reversibly occupy sheath lumen 120 of introducer sheath 100.

In an example, first longitudinal dilator section 410, second longitudinal dilator section 412, and third longitudinal dilator section 416 may each include a blend of 0-95% polypropylene (PP) impact copolymer and 5-100% high performance elastomer. Examples of high performance elastomers may include thermoplastic elastomers such as thermoplastic olefin (TPO), thermoplastic polyurethane (TPU), thermoplastic vulcanizate (TPV), polyester elastomer, polyamide elastomers such as polyester block amide (PEBA), nylon, polyethylene, and fluoropolymers such as fluorinated ethylene propylene (FEP), perfluoroalkoxy alkane (PFA), and ethylene tetrafluoroethylene (ETFE).

Referring to FIG. 5, a distal end view of dilator 400 is illustrated. Distal surface 502 of dilator hub 408 is shown in the background with distal surface 504 of distal tip 406 shown in the foreground. Shaft 402 includes a wall defining dilator lumen 506.

Referring to FIG. 6, a model tracking path 602 on a testing rig 600 used to analyze various medical devices for their abilities to complete turns in vasculature is illustrated. In FIG. 7, the straight lines, numbered in half-integer increments, that intersect model tracking path 602 indicate the numbers of turns through vasculature within model tracking path 602 that a medical device would complete at each straight line, from 0.5 to 5.0 complete turns. Table 1 below provides the average number of turns through model tracking path 602 that each tested introducer sheath and dilator assembly could complete before the introducer sheath and dilator assembly will no longer advance further. As Table 1 indicates, examples of introducer sheaths and dilators of the present disclosure completed an average of 4.7 complete turns, whereas the use of standard introducer sheaths and/or standard dilators resulted in the completion of only 1-2.2 average complete turns. Thus, introducer sheaths and dilators of the present disclosure demonstrate far superior performance of approximately 187.8% over marketed sheaths and dilators.

TABLE 1

| Category | Sheath Model | Dilator | Average # of Turns |
|---|---|---|---|
| Marketed | Sheath including 1 | Supplied Dilator | 2.2 |
| Sheaths | Stiffness Transition | | |
| | Sheath including 2 | Supplied Dilator | 1.7 |
| | Stiffness | | |
| | Transitions | | |
| | Sheath including 0 | Supplied Dilator | 1 |
| | Stiffness | | |
| | Transitions | | |
| Prototype | Introducer Sheath | STD Ansel Dilator | 1.5 |
| | including Sheath | | (8.2% < |
| | Stiffness Transition | | Marketed Sheaths) |
| | | Dilator including | 4.7 |
| | | Dilator Stiffness | (187.8% > |
| | | Transition | Marketed Sheaths) |

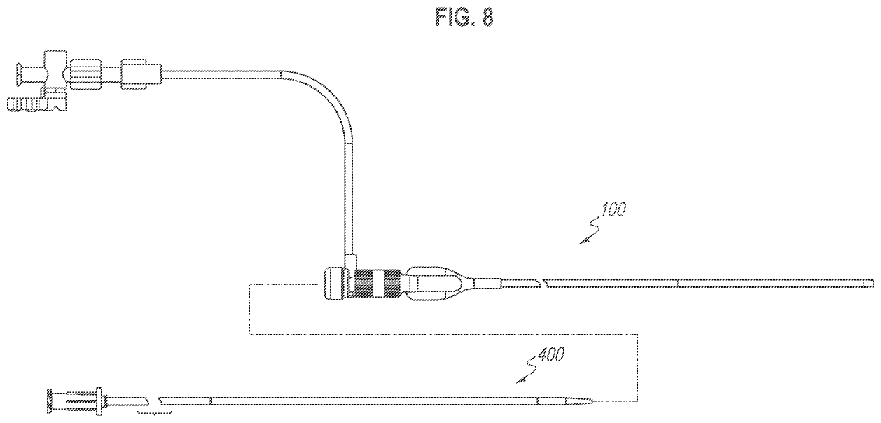

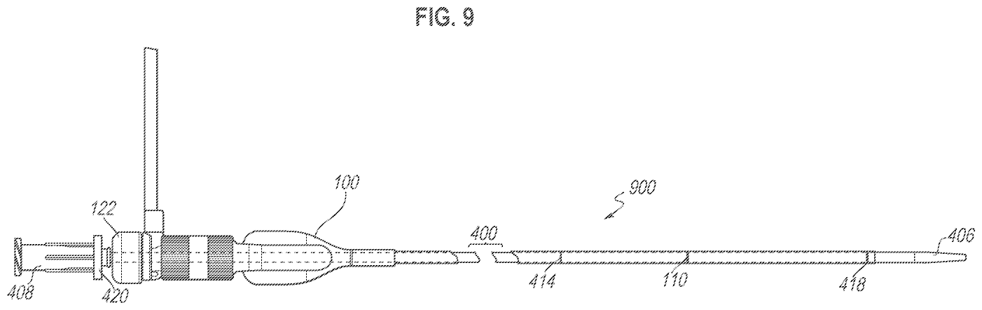

Referring to FIG. 8, a side view of introducer sheath 100 and dilator 400, shown separately, is illustrated. In FIG. 9, a side view of introducer sheath 100 and dilator 400, dilator 400 occupying sheath lumen 120, is illustrated. When distal surface 502 of distal end 420 of dilator hub 408 of dilator 400 confronts or approximates proximal end 122 of valve body 102 of introducer sheath 100, each of first dilator transition 414, sheath transition 110, and second dilator transition 418 are offset longitudinally from one another, and distal tip 406 extends beyond distal tip 112.

Although the present disclosure has been described with reference to examples and the accompanying drawings, the present disclosure is not limited thereto, but may be variously modified and altered by those skilled in the art to which the present disclosure pertains without departing from the spirit and scope of the present disclosure.

The subject-matter of the disclosure may also relate, among others, to the following aspects:

A first aspect relates to a medical device, comprising: an introducer sheath, the introducer sheath comprising a tubular structure comprising a wall defining a sheath lumen extending longitudinally throughout the tubular structure, the tubular structure defining a longitudinal axis therethrough, the tubular structure further comprising: a first longitudinal sheath section having a first sheath stiffness; and a second longitudinal sheath section distal to the first longitudinal sheath section, the second longitudinal sheath section having a second sheath stiffness that is less than the first sheath stiffness; and a sheath transition from the first longitudinal sheath section to the second longitudinal sheath section; and a dilator configured to occupy the sheath lumen, the dilator comprising: a shaft comprising a wall defining a dilator lumen extending longitudinally throughout the shaft, the shaft defining a second longitudinal axis that is coaxial with the longitudinal axis when the dilator occupies the sheath lumen, the shaft further comprising: a first longitudinal dilator section having a first dilator stiffness; and a second longitudinal dilator section distal to the first longitudinal dilator section, the second longitudinal dilator section having a second dilator stiffness that is less than the first dilator stiffness; and a dilator transition from the first longitudinal dilator section to the second longitudinal dilator section; and wherein the sheath transition is offset longitudinally from the dilator transition when the dilator occupies the sheath lumen.

A second aspect relates to the medical device of aspect 1, wherein the first sheath stiffness is less than the first dilator stiffness.

A third aspect relates to the medical device of any preceding aspect, wherein the sheath transition is distal to the dilator transition.

A fourth aspect relates to the medical device of any preceding aspect, wherein the second dilator stiffness is less than the first sheath stiffness and greater than the second sheath stiffness.

A fifth aspect relates to the medical device of aspect 1 or 2, wherein the dilator transition is distal to the sheath transition.

A sixth aspect relates to the medical device of aspect 5, wherein the second dilator stiffness is less than the second sheath stiffness.

A seventh aspect relates to the medical device of any preceding aspect, wherein the second longitudinal sheath section comprises a distal sheath tip at a distal end of the second longitudinal sheath section; and wherein when the dilator occupies the sheath lumen, the second longitudinal dilator section is configured to extend distally at least one centimeter through the distal sheath tip.

An eighth aspect relates to the medical device of any preceding aspect, wherein the shaft further comprises a third longitudinal dilator section distal to the second longitudinal dilator section, the third longitudinal dilator section having a third dilator stiffness different from the second dilator stiffness.

A ninth aspect relates to the medical device of aspect 8, wherein the third dilator stiffness is less than the second dilator stiffness.

A tenth aspect relates to the medical device of aspect 8, wherein the third dilator stiffness is greater than the second dilator stiffness and/or equal to the first dilator stiffness.

An eleventh aspect relates to the medical device of any preceding aspect, wherein a longitudinal dilator length is greater than a longitudinal sheath length.

A twelfth aspect relates to an introducer sheath comprising a tubular structure comprising a wall defining a sheath lumen extending longitudinally throughout the tubular structure, the tubular structure defining a longitudinal axis therethrough, the tubular structure comprising: a first longitudinal sheath section having a first sheath stiffness; a second longitudinal sheath section distal to the first longitudinal sheath section, the second longitudinal sheath section having a second sheath stiffness that is less than the first sheath stiffness; and a sheath transition from the first longitudinal sheath section to the second longitudinal sheath section; wherein a dilator is configured to occupy the sheath lumen such that the sheath transition is offset longitudinally from a dilator transition from a first longitudinal dilator section to a second longitudinal dilator section; and wherein the dilator comprises a shaft, the shaft comprising: a wall defining a dilator lumen extending longitudinally throughout the shaft; the first longitudinal dilator section having a first dilator stiffness; and the second longitudinal dilator section distal to the first longitudinal dilator section and having a second dilator stiffness that is less than the first dilator stiffness.

A thirteenth aspect relates to the introducer sheath of aspect 12, wherein the first sheath stiffness is less than the first dilator stiffness.

A fourteenth aspect relates to the introducer sheath of aspect 12 or 13, wherein the sheath transition is configured to be distal to the dilator transition.

A fifteenth aspect relates to the introducer sheath of any of aspects 12 to 14, wherein the second dilator stiffness is less than the first sheath stiffness and greater than the second sheath stiffness.

A sixteenth aspect relates to the introducer sheath of aspect 12 or 13, wherein the dilator transition is configured to be distal to the sheath transition.

A seventeenth aspect relates to a dilator, comprising a shaft, the shaft comprising: a wall defining a dilator lumen extending longitudinally throughout the shaft; a first longitudinal dilator section having a first dilator stiffness; a second longitudinal dilator section distal to the first longitudinal dilator section, the second longitudinal dilator section having a second dilator stiffness that is less than the first dilator stiffness; and a dilator transition from the first longitudinal dilator section to the second longitudinal dilator section; wherein the dilator is configured to occupy a sheath lumen of an introducer sheath such that a sheath transition from a longitudinal sheath section to a second longitudinal sheath section is offset longitudinally from the dilator transition; and wherein the introducer sheath comprises a tubular structure comprising a wall defining the sheath lumen extending longitudinally throughout the tubular structure, the tubular structure defining a longitudinal axis therethrough, the tubular structure comprising the first longitudinal sheath section having a first sheath stiffness, and the second longitudinal sheath section distal to the first longitudinal sheath section and having a second sheath stiffness that is less than the first sheath stiffness.

An eighteenth aspect relates to the dilator of aspect 17, wherein the first sheath stiffness is less than the first dilator stiffness.

A nineteenth aspect relates to the dilator of aspect 17 or 18, wherein the sheath transition is configured to be distal to the dilator transition.

A twentieth aspect relates to the dilator of aspects 17 to 19, wherein the second dilator stiffness is less than the first sheath stiffness and greater than the second sheath stiffness.

In addition to the features mentioned in each of the independent aspects enumerated above, some examples may show, alone or in combination, the optional features mentioned in the dependent aspects and/or as disclosed in the description above and shown in the figures.

What is claimed is:

1. A medical device, comprising:
an introducer sheath, the introducer sheath comprising a tubular structure comprising a wall defining a sheath lumen extending longitudinally throughout the tubular structure, the tubular structure defining a longitudinal axis therethrough, the tubular structure further comprising:
a first longitudinal sheath section having a first sheath stiffness; and
a second longitudinal sheath section distal to the first longitudinal sheath section, the second longitudinal sheath section having a second sheath stiffness that is less than the first sheath stiffness; and
a sheath transition from the first longitudinal sheath section to the second longitudinal sheath section; and
a dilator configured to occupy the sheath lumen, the dilator comprising:
a shaft comprising a wall defining a dilator lumen extending longitudinally throughout the shaft, the shaft defining a second longitudinal axis that is coaxial with the longitudinal axis when the dilator occupies the sheath lumen, the shaft further comprising:
a first longitudinal dilator section having a first dilator stiffness; and
a second longitudinal dilator section distal to the first longitudinal dilator section, the second longitudinal dilator section having a second dilator stiffness that is less than the first dilator stiffness; and
a dilator transition from the first longitudinal dilator section to the second longitudinal dilator section; and
wherein the sheath transition is offset longitudinally from the dilator transition when the dilator occupies the sheath lumen.

2. The medical device of claim 1, wherein the first sheath stiffness is less than the first dilator stiffness.

3. The medical device of claim 1, wherein the sheath transition is distal to the dilator transition.

4. The medical device of claim 3, wherein the second dilator stiffness is less than the first sheath stiffness and greater than the second sheath stiffness.

5. The medical device of claim 1, wherein the dilator transition is distal to the sheath transition.

6. The medical device of claim 5, wherein the second dilator stiffness is less than the second sheath stiffness.

7. The medical device of claim 1, wherein the second longitudinal sheath section comprises a distal sheath tip at a distal end of the second longitudinal sheath section; and
wherein when the dilator occupies the sheath lumen, the second longitudinal dilator section is configured to extend distally at least one centimeter through the distal sheath tip.

8. The medical device of claim 1, wherein the shaft further comprises a third longitudinal dilator section distal to the second longitudinal dilator section, the third longitudinal dilator section having a third dilator stiffness different from the second dilator stiffness.

9. The medical device of claim 8, wherein the third dilator stiffness is less than the second dilator stiffness.

10. The medical device of claim 8, wherein the third dilator stiffness is greater than the second dilator stiffness and/or equal to the first dilator stiffness.

11. The medical device of claim 1, wherein a longitudinal dilator length is greater than a longitudinal sheath length.

12. An introducer sheath comprising a tubular structure comprising a wall defining a sheath lumen extending longitudinally throughout the tubular structure, the tubular structure defining a longitudinal axis therethrough, the tubular structure comprising:
a first longitudinal sheath section having a first sheath stiffness;
a second longitudinal sheath section distal to the first longitudinal sheath section, the second longitudinal sheath section having a second sheath stiffness that is less than the first sheath stiffness; and
a sheath transition from the first longitudinal sheath section to the second longitudinal sheath section;
wherein a dilator is configured to occupy the sheath lumen such that the sheath transition is offset longitudinally from a dilator transition from a first longitudinal dilator section to a second longitudinal dilator section; and
wherein the dilator comprises a shaft, the shaft comprising: a wall defining a dilator lumen extending longitudinally throughout the shaft; the first longitudinal dilator section having a first dilator stiffness; and the second longitudinal dilator section distal to the first longitudinal dilator section and having a second dilator stiffness that is less than the first dilator stiffness.

13. The introducer sheath of claim 12, wherein the first sheath stiffness is less than the first dilator stiffness.

14. The introducer sheath of claim 12, wherein the sheath transition is configured to be distal to the dilator transition.

15. The introducer sheath of claim 14, wherein the second dilator stiffness is less than the first sheath stiffness and greater than the second sheath stiffness.

16. The introducer sheath of claim 12, wherein the dilator transition is configured to be distal to the sheath transition.

17. A dilator, comprising a shaft, the shaft comprising:
a wall defining a dilator lumen extending longitudinally throughout the shaft;
a first longitudinal dilator section having a first dilator stiffness;
a second longitudinal dilator section distal to the first longitudinal dilator section, the second longitudinal dilator section having a second dilator stiffness that is less than the first dilator stiffness; and
a dilator transition from the first longitudinal dilator section to the second longitudinal dilator section;
wherein the dilator is configured to occupy a sheath lumen of an introducer sheath such that a sheath transition from a first longitudinal sheath section to a second longitudinal sheath section is offset longitudinally from the dilator transition; and
wherein the introducer sheath comprises a tubular structure comprising a wall defining the sheath lumen extending longitudinally throughout the tubular structure, the tubular structure defining a longitudinal axis therethrough, the tubular structure comprising the first longitudinal sheath section having a first sheath stiffness, and the second longitudinal sheath section distal to the first longitudinal sheath section and having a second sheath stiffness that is less than the first sheath stiffness.

18. The dilator of claim 17, wherein the first sheath stiffness is less than the first dilator stiffness.

19. The dilator of claim 17, wherein the sheath transition is configured to be distal to the dilator transition.

20. The dilator of claim 19, wherein the second dilator stiffness is less than the first sheath stiffness and greater than the second sheath stiffness.
